(19) United States
(12) Patent Application Publication
(10) Pub. No.: US 2025/0095797 A1
(43) Pub. Date: Mar. 20, 2025

(54) METHOD, INFORMATION PROCESSING DEVICE, AND RECORDING MEDIUM FOR PERFORMING PREDICTION RELATED TO ADDITION POLYMERIZATION REACTION

(71) Applicant: DIC Corporation, Tokyo (JP)

(72) Inventors: Atsushi Nagao, Osaka (JP); Yoshimasa Matsufuji, Osaka (JP); Naoki Shibamoto, Osaka (JP); Shinsuke Kariura, Tokyo (JP); Kouichi Matsui, Chiba (JP)

(73) Assignee: DIC Corporation, Tokyo (JP)

(21) Appl. No.: 18/566,769

(22) PCT Filed: May 22, 2023
(86) PCT No.: PCT/JP2023/019014

§ 371 Date: Dec. 4, 2023

(30) Foreign Application Priority Data
Mar. 24, 2023 (JP) 2023-048922

(51) Int. Cl. G16C 60/00; C08F 20/00; G06N 3/08; G16C 20/10; G16C 20/70

(52) U.S. Cl. CPC G16C 60/00; C08F 20/00; G06N 3/08; G16C 20/10; G16C 20/70

(57) ABSTRACT

A method for performing prediction related to an addition polymerization reaction is executed by an information processing device and includes: training a prediction model based on actual data including a plurality of explanatory factors and an objective factor that are related to the addition polymerization reaction; and predicting, with the prediction model, the objective factor during the addition polymerization reaction based on the explanatory factors related to the addition polymerization reaction. The explanatory factors include a plurality of feature values obtained by a clustering analysis of time-series data from a plurality of measurement instruments at a temperature rising process and a dropwise addition process, and the objective factor includes at least either a nonvolatile content (NV) or a solution viscosity.

### CROSS-REFERENCE TO RELATED APPLICATIONS

The present application claims priority from Japanese Patent Application No. 2023-048922, filed on Mar. 24, 2023, the contents of which are incorporated herein by reference.

### BACKGROUND

### Technical Field

The present disclosure relates to a method for performing prediction related to an addition polymerization reaction, an information processing device, and a recording medium storing instructions.

### Description of Related Art

Conventionally, methods for performing prediction related to chemical reactions have been developed (for example, PTL 1).

### PATENT LITERATURE

PTL 1: WO 2003/026791

The technique described in PTL 1 has described the use of modeling techniques such as neural networks, partial least squares, and principal component regression to optimize the control of reactor systems. However, specific design methods and optimization in performing predictions related to addition polymerization reactions have not been considered, and there has been room for improvement in the prediction technology related to the addition polymerization reactions.

### SUMMARY

One or more embodiments of the present disclosure made in view of such circumstances improve the prediction technology related to the addition polymerization reactions.

(1) A method in one or more embodiments of the present disclosure is a method for performing prediction related to an addition polymerization reaction executed by an information processing device, the method comprising:
a step of training a prediction model based on actual data comprising a plurality of explanatory factors and an objective factor that are related to the addition polymerization reaction; and
a step of predicting, with the prediction model, the objective factor during the addition polymerization reaction based on the explanatory factors related to the addition polymerization reaction, in which
the explanatory factors include a plurality of feature values obtained by a clustering analysis of time-series data from a plurality of measurement instruments at a temperature rising process and a dropwise addition process, and
the objective factor includes at least either a nonvolatile content (NV) or a solution viscosity.

(2) The method in one or more embodiments of the present disclosure is the method according to (1), in which the explanatory factors include a theoretical value in a reaction physics model.

(3) The method in one or more embodiments of the present disclosure is the method according to (1) or (2), in which the addition polymerization reaction is an addition polymerization reaction of an acrylic.

(4) The method in one or more embodiments of the present disclosure is the method according to any one of (1) to (3), in which the prediction model is a neural network model including: an input layer; an intermediate layer; and an output layer, and a coefficient of an activation function of the intermediate layer is larger than a coefficient of an activation function of the output layer.

(5) An information processing device in one or more embodiments of the present disclosure is an information processing device performing prediction related to an addition polymerization reaction, the information processing device comprising: a control unit that:
trains a prediction model based on actual data comprising a plurality of explanatory factors and an objective factor that are related to the addition polymerization reaction, and
predicts, with the prediction model, the objective factor during the addition polymerization reaction based on the explanatory factors related to the addition polymerization reaction, and
the explanatory factors include a plurality of feature values obtained by a clustering analysis of time-series data from a plurality of measurement instruments at a temperature rising process and a dropwise addition process, and
the objective factor includes at least either a nonvolatile content (NV) or a solution viscosity.

(6) A non-transitory computer-readable recording medium according to one or more embodiments of the present disclosure is a non-transitory computer-readable recording medium storing instructions performing prediction related to an addition polymerization reaction by an information processing device that comprises a processor, the instructions causing the processor to execute:
training a prediction model based on actual data comprising a plurality of explanatory factors and an objective factor that are related to the addition polymerization reaction; and
predicting, with the prediction model, the objective factor during the addition polymerization reaction based on the explanatory factors related to the addition polymerization reaction,
the explanatory factors include a plurality of feature values obtained by a clustering analysis of time-series data from a plurality of measurement instruments at a temperature rising process and a dropwise addition process, and
the objective factor includes at least either a nonvolatile content (NV) or a solution viscosity.

According to the method for performing prediction related to an addition polymerization reaction, the information processing device, and the recording medium storing instructions in one or more embodiments of the present disclosure, the prediction technology related to the addition polymerization reaction can be improved.

### BRIEF DESCRIPTION OF DRAWINGS

FIG. 1 is a block diagram illustrating the schematic configuration of an information processing device according to one or more embodiments.

FIG. 2 is a flowchart illustrating operations of the information processing device according to one or more embodiments.

FIG. 3 is concept of an addition polymerization reaction process according to one or more embodiments.

FIG. 4A is a view illustrating the time transition of each piece of data pertaining to a temperature rising process and a dropwise addition process.

FIG. 4B illustrates the time transition of categories related to the temperature rising process and the dropwise addition process.

FIG. 5 illustrates one example of the accuracy verification result of a prediction model according to one or more embodiments.

FIG. 6 illustrates one example of the accuracy verification result of the prediction model according to one or more embodiments.

FIG. 7 is one example of the conceptual view of the prediction model in one or more embodiments.

### DETAILED DESCRIPTION OF EMBODIMENTS

Hereinafter, a method for performing prediction related to an addition polymerization reaction, an information processing device, and a recording medium storing instructions in one or more embodiments of the present disclosure will be described with reference to the drawings. A prediction target according to one or more embodiments of the present disclosure includes both batch reaction and continuous reaction. Examples of the main polymer materials synthesized by the addition polymerization reaction according to one or more embodiments include acrylic, poly(meth)acrylic acid esters, polyethylene, polypropylene, polystyrene, polyvinyl chloride, polyvinyl acetate, polyvinylidene chloride, polyacrylonitrile, and polytetrafluoroethylene. For example, the addition polymerization reaction according to one or more embodiments includes an addition polymerization reaction of the acrylic.

In each of the drawings, identical or equivalent parts are assigned the same symbols. In the description of one or more embodiments, descriptions of the identical or equivalent parts are omitted or simplified as appropriate.

First, the overview of one or more embodiments will be described. The method for performing prediction related to an addition polymerization reaction in one or more embodiments is executed by an information processing device 10. The information processing device 10 trains a prediction model based on actual data including a plurality of explanatory factors and an objective factor related to the addition polymerization reaction. The information processing device 10 predicts the objective factor during the addition polymerization reaction based on the explanatory factors related to the addition polymerization reaction by the trained prediction model. The explanatory factors include a plurality of feature values obtained by clustering analysis of time-series data from a plurality of measurement instruments at a temperature rising process and a dropwise addition process. The objective factor is characterized by including at least either an NV or a solution viscosity. The NV stands for Nonvolatile content or Non-volatile matter content and represents the nonvolatile content in a resin solution. The addition polymerization reaction is basically synthesized in a solvent. A polymerized polymer is considered the nonvolatile content, whereas a non-polymerized monomer is considered a volatile content. As the addition polymerization reaction proceeds and the monomer is consumed, the volatile content decreases and the nonvolatile content increases. Checking an increase in the nonvolatile content to a certain value allows a reaction ratio to be estimated.

As described above, according to one or more embodiments, the explanatory factors include the feature values obtained by the clustering analysis of the time-series data from the measurement instruments at the temperature rising process and the dropwise addition process. The objective factor is characterized by including either an NV or a solution viscosity. In the case where such an objective factor in the addition polymerization reaction is predicted, the prediction accuracy can be improved by including, in the explanatory factors, the feature values obtained by the clustering analysis of the time-series data from the measurement instruments at the temperature rising process and the dropwise addition process as described later. Therefore, according one or more embodiments, the prediction technology related to the addition polymerization reaction can be improved.

### (Configuration of Information Processing Device)

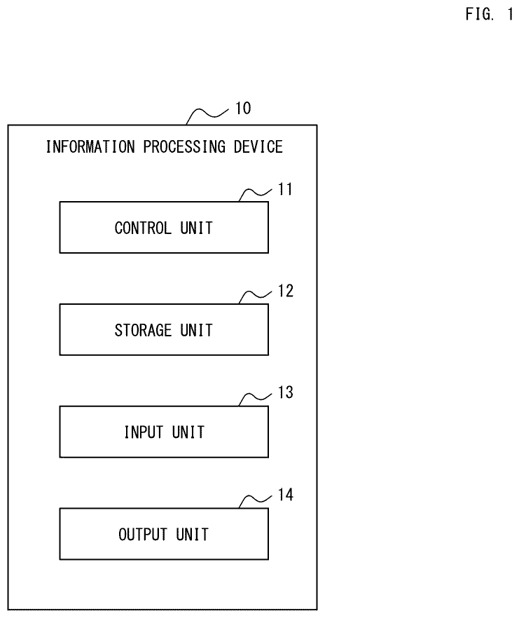

Subsequently, referring to FIG. 1, each configuration of the information processing device 10 will be described in detail. The information processing device 10 is an arbitrary device used by users. For example, personal computers, server computers, general-purpose electronic devices, or dedicated electronic devices can be employed as the information processing device 10.

As illustrated in FIG. 1, the information processing device 10 includes a control unit (or a processor) 11, a storage unit (or a storage) 12, an input unit (or an input interface) 13, and an output unit (or an output interface) 14.

The control unit 11 includes at least one processor, at least one dedicated circuit, or a combination thereof. The processor is a general-purpose processor such as a central processing unit (CPU) or a graphics processing unit (GPU), or a dedicated processor specialized for specific processing. The dedicated circuit is, for example, a field-programmable gate array (FPGA) or an application specific integrated circuit (ASIC). The control unit 11 executes processes associated with the operation of the information processing device 10 while controlling each part of the information processing device 10.

The storage unit 12 includes at least one semiconductor memory, at least one magnetic memory, at least one optical memory, or a combination of at least two of these memories. The semiconductor memory is, for example, a random-access memory (RAM) or a read-only memory (ROM). The RAM is, for example, a static random-access memory (SRAM) or a dynamic random-access memory (DRAM). The ROM is, for example, an electrically erasable programmable read-only memory (EEPROM). The storage unit 12 functions, for example, as a main memory device, an auxiliary memory device, or a cache memory. In the storage unit 12, data used in the operation of the information processing device 10 and data obtained by the operation of the information processing device 10 are stored.

The input unit 13 includes at least one interface for input. The interface for input is, for example, physical keys, capacitive keys, pointing devices, or touch screens integrated with displays. The interface for input may be, for example, a microphone that accepts voice input or a camera that accepts gesture input. The input unit 13 accepts operations to input data used in the operation of the information processing device 10. The input unit 13 may be connected to the information processing device 10 as an external input device instead of being provided in the information processing device 10. For example, any method such as universal serial bus (USB), high-definition multimedia interface (HDMI) (registered trademark), or Bluetooth (registered trademark) can be used as the connection method.

The output unit 14 includes at least one interface for output. The interface for output is, for example, a display that outputs information in the form of images. The display is, for example, a liquid crystal display (LCD) or an organic electroluminescence (EL) display. The output unit 14 displays and outputs data obtained by the operation of the information processing device 10. The output unit 14 may be connected to the information processing device 10 as an external output device instead of being provided in the information processing device 10. For example, any method such as USB, HDMI (registered trademark), or Bluetooth (registered trademark) can be used as the connection method.

The functions of the information processing device 10 are achieved by executing a program or instructions according to one or more embodiments on a processor corresponding to the information processing device 10. In other words, the functions of the information processing device 10 are achieved by software. The instructions cause the computer to function as the information processing device 10 by causing the computer to execute the operations of the information processing device 10. In other words, the computer functions as the information processing device 10 by executing the operation of the information processing device 10 in accordance with the instructions.

In one or more embodiments, the instructions can be recorded on a computer-readable recording medium. The computer-readable recording media include non-transient computer-readable media, for example, magnetic recording devices, optical discs, magneto-optical recording media, or semiconductor memories.

Some or all of the functions of the information processing device 10 may be achieved by a dedicated circuit corresponding to the control unit 11. In other words, some or all of the functions of the information processing device 10 may be achieved by hardware.

In one or more embodiments, the storage unit 12 stores therein, for example, actual data and prediction models. The actual data and the prediction model may be stored in an external device separate from the information processing device 10. In this case, the information processing device 10 may be equipped with an interface for external communication. The interface for communication may be either an interface of a wired communication or an interface of wireless communication. In the case of the wired communication, the interface for communication is, for example, a LAN interface or USB. In the case of the wireless communication, the interface for communication is, for example, an interface compliant with mobile communication standards such as LTE, 4G, or 5G, or an interface compliant with short-range wireless communication such as Bluetooth (registered trademark). The interface for communication can receive data used in the operation of the information processing device 10 and can transmit data obtained by the operation of the information processing device 10.

### (Operation of Information Processing Device)

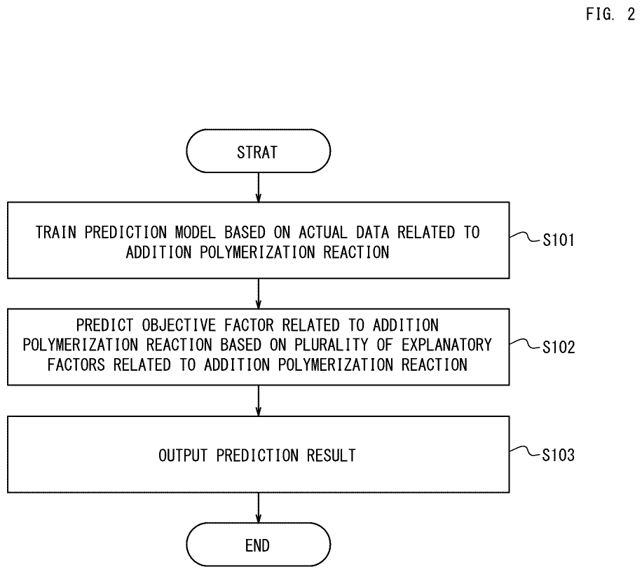

Subsequently, with reference to FIG. 2, the operation of the information processing device 10 according to one or more embodiments will be described.

Step S101: The control unit 11 of the information processing device 10 trains a prediction model based on actual data on the addition polymerization reaction. The actual data include the explanatory factors and the objective factor related to the addition polymerization reaction. The explanatory factors include the feature values obtained by the clustering analysis of the time-series data from the measurement instruments at the temperature rising process and the dropwise addition process. The objective factor includes at least either an NV or a solution viscosity. In other words, the control unit 11 trains the prediction model using these explanatory factors and objective factor included in the actual data as learning data.

Any method can be employed to acquire the actual data. For example, the control unit 11 acquires the actual data from the storage unit 12. The control unit 11 may also acquire the actual data by accepting input of the actual data from the user by the input unit 13. Alternatively, the control unit 11 may acquire such actual data from an external device that stores therein the actual data through an interface for communication.

The prediction model trained based on the learning data is cross-validated. As a result of such cross-validation, in the case where an accuracy is within a practical range, the prediction related to the addition polymerization reaction is performed using the prediction model.

Step S102: The control unit 11 predicts the objective factor related to the addition polymerization reaction based on the explanatory factors related to the addition polymerization reaction. For example, the control unit 11 may acquire the explanatory factors by accepting input of the explanatory factors from the user by the input unit 13.

Step S103: The control unit 11 outputs the objective factor predicted at Step S102 as the prediction result from the output unit 14.

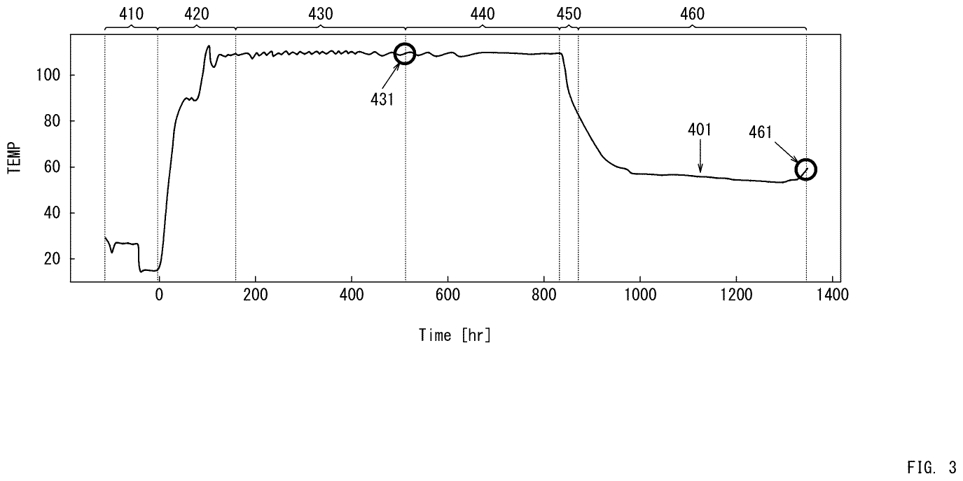

Here, in one or more embodiments, the explanatory factors are characterized by including the feature values obtained by the clustering analysis of the time-series data from the measurement instruments at the temperature rising process and the dropwise addition process. FIG. 3 is a conceptual view illustrating the addition polymerization reaction process. As illustrated in FIG. 3, the addition polymerization reaction includes a charging process 410, a temperature rising process 420, a dropwise addition process 430, a holding process 440, a cooling process 450, and a post-cooling process 460. A graph 401 illustrates the temperature transition of a material to be synthesized. At the temperature rising process 420, the temperature of the material to be synthesized rises. In the holding process 440, the temperature of the material to be synthesized is kept constant. At a final stage 461 of the post-cooling process 460, the quality values of the material are analyzed.

The above analytical values in the addition polymerization reaction correspond to the objective factor in one or more embodiments. Such analytical values also depend on the temperature rising process and the dropwise addition process. On the other hand, a wide variety of time-series data are involved at the temperature rising process and the dropwise addition process, and thus using all of these time-series data as the explanatory variables is not realistic. Here, the time-series data include measurement values at intervals of 1 second to 1 minute. Therefore, one or more embodiments are characterized by using the feature values obtained by the clustering analysis of the time-series data from the measurement instruments at the temperature rising process and the dropwise addition process as the explanatory factors. Specifically, the clustering analysis is performed at the end of the dropwise addition process 430 in FIG. 3 and, the reaction prediction is performed all at once for approximately 5 hours (the time of the holding process 440) ahead to determine the cooling timing in the cooling process 450 from the timing (at the end point of the dropwise addition process 431) when all the explanatory factors are collected.

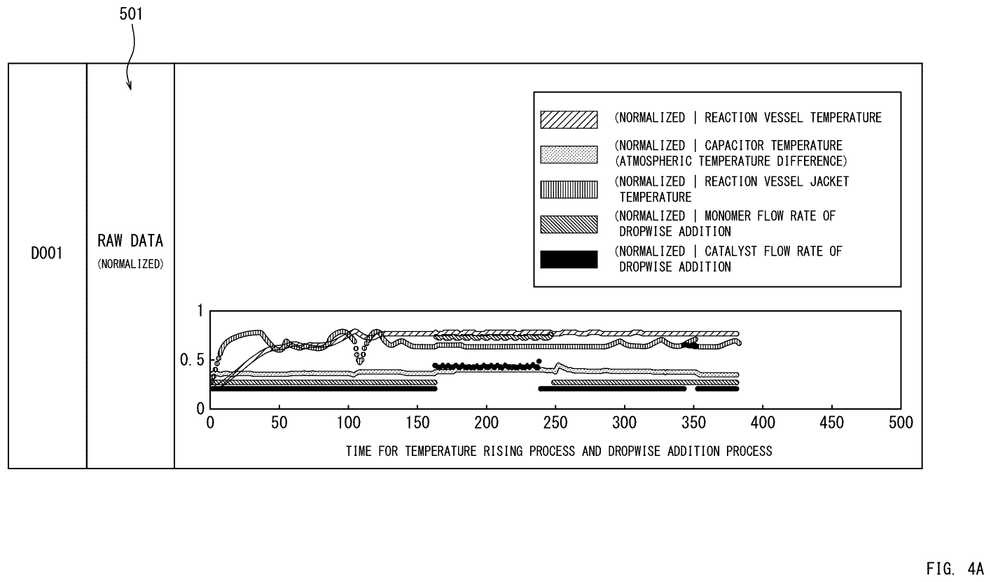

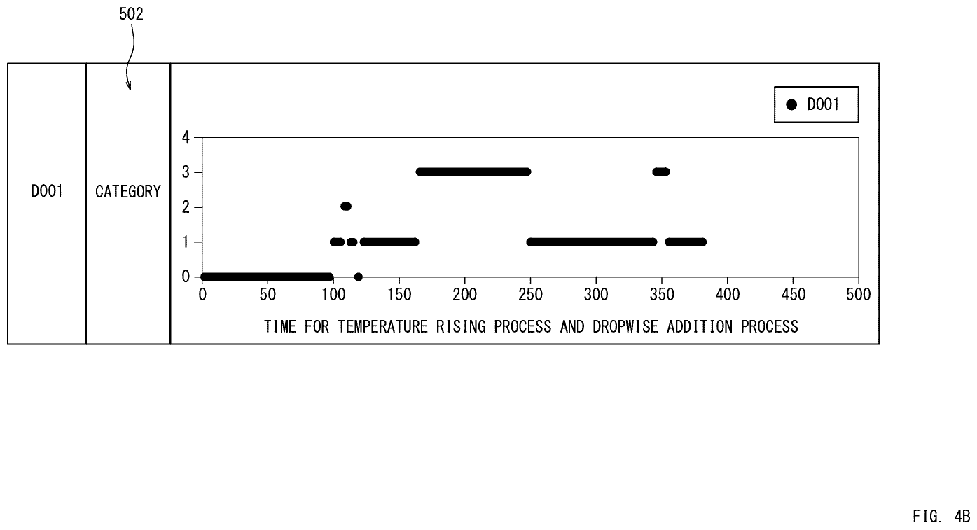

FIG. 4A and FIG. 4B illustrate a method for calculating the feature values in a certain lot (a lot having a lot number of D001). An item 501 in FIG. 4A represents the time transition of each piece of the data pertaining to the temperature rising process and the dropwise addition process related to such a lot. Such data include data on a reaction vessel temperature, a capacitor temperature (atmospheric temperature difference), a reaction vessel jacket temperature, a monomer flow rate of dropwise addition, and a catalyst flow rate of dropwise addition. Each piece of the data in the item 501 is normalized. An item 502 in FIG. 4B represents the time transition of the categories related to the temperature rising process and the dropwise addition process. In one or more embodiments, the categories include five steps of 0-4. Specifically, the categories are determined by the clustering analysis of the time-series data from the measurement instruments at the temperature rising process and the dropwise addition process. Based on these categories, the feature values at the temperature rising process and the dropwise addition process are determined. For example, the feature values are determined based on the time rate of the categories. The time rate of the categories refers to a value obtained by dividing the cumulative residence time of each category by the overall time. As described above, in one or more embodiments, the feature values are provided by the clustering analysis at the temperature rising process and the dropwise addition process.

As described above, according to one or more embodiments, the explanatory factors include the feature values obtained by the clustering analysis of the time-series data from the measurement instruments at the temperature rising process and the dropwise addition process. The objective factor is characterized by including either an NV or a solution viscosity. In the case where the prediction related to the addition polymerization reaction is performed, the accuracy of the prediction model can be improved by including, in the explanatory factors, the feature values obtained by the clustering analysis of the temperature rising process and the dropwise addition process. Therefore, according one or more embodiments, the prediction technology related to the addition polymerization reaction can be improved.

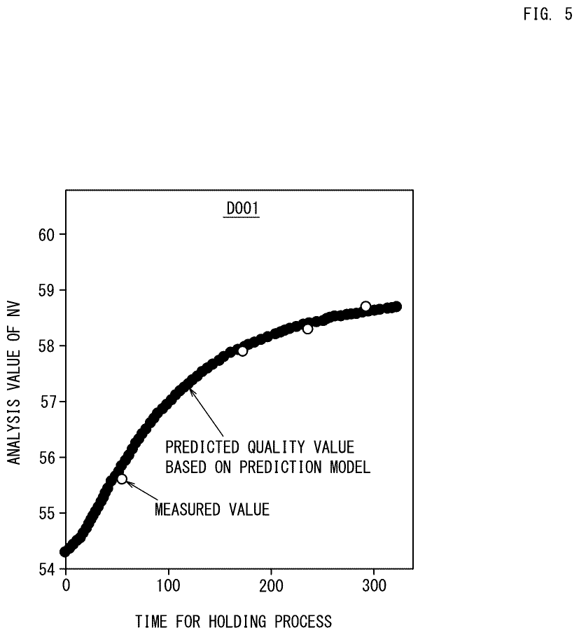

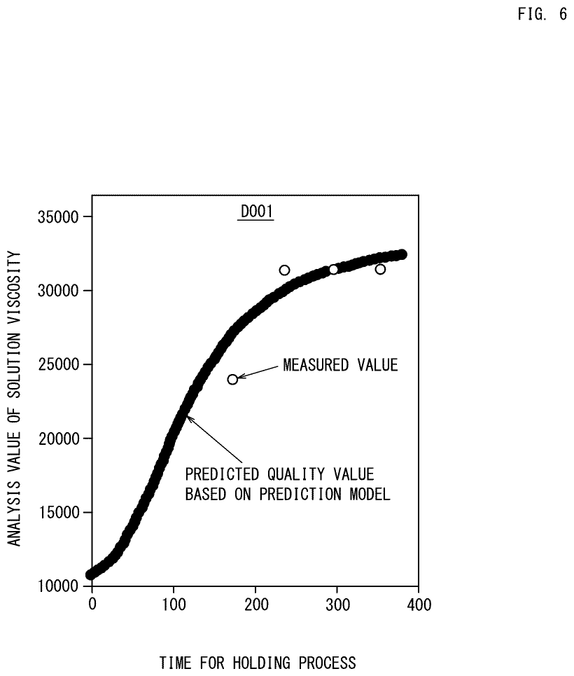

FIG. 5 and FIG. 6 illustrate an example of the accuracy verification results of the prediction model according to one or more embodiments. FIG. 5 is a graph illustrating the NV and the measured values of a certain lot (lot number D001) of an acrylic predicted by the prediction model. As illustrated in FIG. 5, the NV predicted by the prediction model approximately agrees with the measured values. FIG. 6 is a graph illustrating the solution viscosity and the measured values of the above lot of the acrylic predicted by the prediction model. As illustrated in FIG. 6, the solution viscosity predicted by the prediction model approximately agrees with the measured values. Therefore, it is found that the accuracy of the prediction model is sufficiently high.

Here, the explanatory factors may include theoretical values in a reaction physics model (reaction physics model theoretical values). As described above, the reaction physics model may serve as explanatory factors as a baseline of the reaction characteristics. Similarly, the explanatory factors may include the amount of a charged raw material monomer, the amount of a charged raw material solvent, the amount of a charged initiator, the reaction vessel temperature, the capacitor temperature, the reaction vessel jacket temperature, and the like.

Here, the prediction model according to one or more embodiments may be, for example, a neural network model. In the case where the prediction model is the neural network model, the coefficients of activation functions of the neural network model may be different between an intermediate layer and an output layer. For example, the coefficient of the activation function of the intermediate layer is larger than the coefficient of the activation function of the output layer.

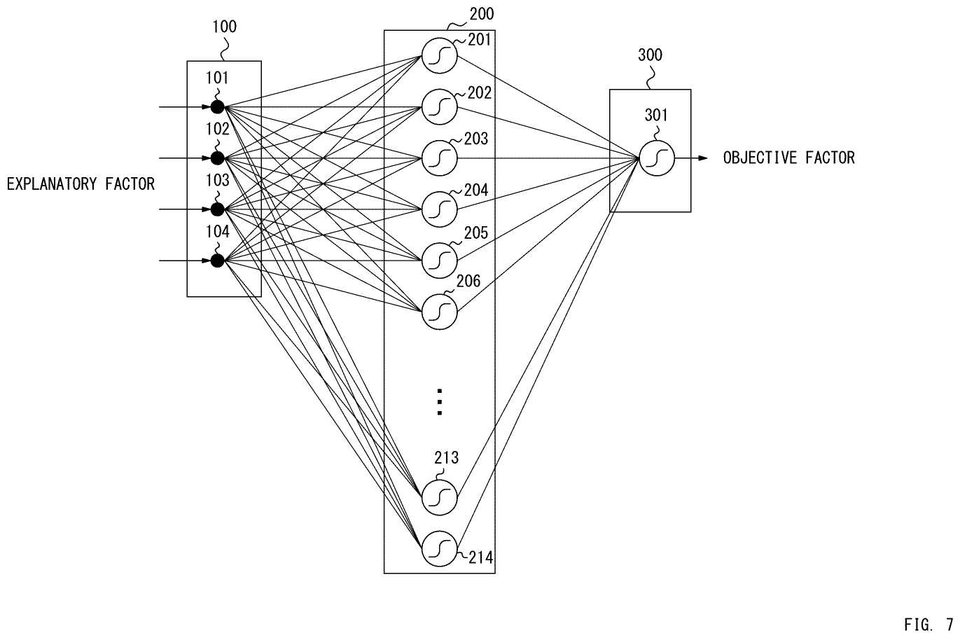

FIG. 7 is a conceptual diagram of the neural network model according to one or more embodiments. Such a neural network model includes an input layer 100, an intermediate layer 200, and an output layer 300. The neural network model in one or more embodiments is fully connected. In one or more embodiments, the number of layers in the neural network model is, for example, 2. Such number of layers is the number of layers excluding the input layer. By setting the number of layers in the neural network model to 2, a model configuration can be prevented from becoming inappropriate to the physical phenomena in the addition polymerization reaction. In other words, the number of layers of neural network model can be kept to minimum necessary, whereby the model configuration suitable for the physical phenomena in the addition polymerization reaction can be achieved. The number of layers of the neural network model according to one or more embodiments is not limited to this number of layers, and may be three layers or more. In the case where the number of layers in the neural network model is three or more, as the layer of the neural network model becomes more front side, the coefficient of the activation function may be set larger.

The input layer 100 includes a plurality of elements 101 to 104 (also referred to as input elements 101 to 104). In the neural network model illustrated in FIG. 7, the number of the input elements is 4. The input elements 101 to 104 are also referred to as the first to fourth elements, respectively. In the input elements 101 to 104, each of the explanatory factors is input. The number of input elements is not limited to this and may be less than 4 or 5 or more.

The intermediate layer 200 includes a plurality of elements 201 to 214 (also referred to as intermediate elements 201 to 214). In the neural network model illustrated in FIG. 7, the number of the intermediate elements is 14. The intermediate elements 201 to 214 are also referred to as the first to fourteenth elements, respectively. The number of intermediate elements is not limited to this, and may be less than 14 or 15 or more.

The output layer 300 includes an element 301 (an output element 301). In the neural network model illustrated in FIG. 7, the number of the output elements is 1. The output element 301 is also referred to as the first element The number of the output elements is not limited to this, and may be 2 or more.

The values input from the input elements 101 to 104 of the input layer 100 to the intermediate elements 201 to 214 of the intermediate layer 200 are converted in the intermediate layer 200 based on the activation function of the intermediate layer 200. The converted values are output to the element 301 of the output layer 300. The activation function of the intermediate layer 200 is, for example, a sigmoid function. The values input from the intermediate elements 201 to 214 of the intermediate layer 200 to the output element 301 of the output layer 300 are converted in the output layer 300 based on the activation function of the output layer 300 and output. The activation function of the output layer 300 is, for example, the sigmoid function. Specifically, the activation functions of the intermediate layer and the output layer are, for example, the respective sigmoid functions determined by the following formulas (1) and (2).

$$\begin{matrix}[ Mathematical Formula 1 ] & \\ {f}^{1} ( {u}_{j}^{1} ) = \frac{1}{1 + {e}^{- {a}_{1} {u}_{j}^{1}}} & ( 1 )\end{matrix}$$

$${f}^{2} ( {u}_{j}^{2} ) = \frac{1}{1 + {e}^{- {a}_{2} {u}_{j}^{2}}} \qquad ( 2 )$$

Here, f¹(uⱼ¹) is the activation function of the intermediate layer 200, a₁ is the coefficient of the activation function of the intermediate layer 200, and uⱼ¹ is the input value input to the j-th element of the intermediate layer 200. In the example in FIG. 7, the number of the intermediate elements is 14, and this j takes the value from 1 to 14. f²(uⱼ²) is the activation function of the output layer 300, a₂ is the coefficient of the activation function of the output layer 300, and uⱼ² is the input value input to the j-th element of the output layer 300. In the example in FIG. 7, j is 1 because the number of the output elements is 1. As described above, in the neural network model according to one or more embodiments, the coefficient of the activation function of the intermediate layer 200 is larger than the coefficient of the activation function of the output layer 300. In other words, a₁ and a₂ in the neural network model according to one or more embodiments satisfy a₁>a₂.

In the neural network model according to one or more embodiments, the coefficient of the activation function of the intermediate layer is larger than the coefficient of the activation function of the output layer. This allows the configuration of the neural network model to be optimized at the time of performing the prediction related to the addition polymerization reaction. Specifically, in the neural network model for performing the prediction related to the addition polymerization reaction, change in the explanatory factors is desirably viewed as obvious change. Therefore, by setting the coefficient of the activation functions of the intermediate layer larger than the coefficient of the activation functions of the output layer, the change in the input values to the intermediate layer can be transmitted to the output layer as the obvious change. On the other hand, in the output layer of the neural network model for performing the prediction related to the addition polymerization reaction, the values of the training data and the objective factor are required to be converged. Therefore, the coefficient of the activation function of the output layer is set smaller than the coefficient of the activation function of the intermediate layer. By doing so, the value of the objective factor output from the output layer is finely adjusted.

By setting the coefficients of the activation functions between the intermediate layer and the output layer to be different, the learning process of the neural network model is optimized. Specifically, the updated amounts of the weight variables in the output layer and the intermediate layer during the learning process can be adjusted by changing the coefficient of the activation function. In addition, updating the weight variables provides a significant impact on the learning process. Therefore, the learning process may be optimized based on the adjustment of the updated amounts.

Specifically, in the neural network model at the time of performing prediction related to the addition polymerization reaction, the updated amount of the weight variables in the intermediate layer may be set to be relatively large. This allows the weight variables in the intermediate layer to vary more significantly during the learning process, and thus changes in the input values to the intermediate layer to be transferred to the output layer as obvious changes. On the other hand, the updated amount of weight variables in the output layer may be set to be relatively small. This allows the weight variables in the output layer to vary less during the learning process and thus the values of the training data and the objective factor to be easily converged. In addition, by satisfying a^{l}>a^{L}, an arbitrary smooth function can be approximated with sufficient accuracy, eliminating the need to inadvertently increase the number of intermediate layers. This allows sufficient accuracy to be obtained even when the intermediate layer is one layer. Preparing fewer intermediate layers directly leads to reduction in generation of over-fitting and thus provides a secondary effect on stability of the learning process and, in addition, robustness of the model.

The case where the activation functions of the intermediate layer and the output layer are sigmoid functions is described in one or more embodiments. However, the activation functions are not limited to the sigmoid functions. For example, the activation functions of the intermediate layer and the output layer may be functions such as a hyperbolic tangent function (tan h function) and a ramp functions (ReLU).

Although the present disclosure has been described based on the drawings and examples, it should be noted that those skilled in the art can easily make changes and modifications based on the present disclosure. Therefore, it should be noted that these changes and modifications are included within the scope of the present disclosure. For example, the functions and the like included in the units, steps, or the like can be rearranged so as not to be logically inconsistent, and a plurality of units, steps, or the like can be combined to one or divided.

### REFERENCE SIGNS LIST

10 information processing device
11 control unit
12 storage unit
13 input unit
14 output unit
100 input layer
200 intermediate layer
300 output layer
101 to 104, 201 to 214, and 301 element
401 graph
410 charging process
420 temperature rising process
430 dropwise addition process
431 end point of temperature rising process
440 holding process
450 cooling process
460 post-cooling process
461 final stage
501 and 502 item

1. A method for performing prediction related to an addition polymerization reaction, the method being executed by an information processing device and comprising:
training a prediction model based on actual data comprising a plurality of explanatory factors and an objective factor that are related to the addition polymerization reaction; and
predicting, with the prediction model, the objective factor during the addition polymerization reaction based on the explanatory factors related to the addition polymerization reaction, wherein
the explanatory factors include a plurality of feature values obtained by a clustering analysis of time-series data from a plurality of measurement instruments at a temperature rising process and a dropwise addition process, and
the objective factor includes at least either a nonvolatile content (NV) or a solution viscosity.

2. The method according to claim 1, wherein the explanatory factors include a theoretical value in a reaction physics model.

3. The method according to claim 1, wherein the addition polymerization reaction is an addition polymerization reaction of an acrylic.

4. The method according to claim 1, wherein
the prediction model is a neural network model including;
an input layer;
an intermediate layer; and
an output layer, and
a coefficient of an activation function of the intermediate layer is larger than a coefficient of an activation function of the output layer.

5. An information processing device performing prediction related to an addition polymerization reaction, the information processing device comprising:
a control unit that:
trains a prediction model based on actual data comprising a plurality of explanatory factors and an objective factor that are related to the addition polymerization reaction, and
predicts, with the prediction model, the objective factor during the addition polymerization reaction based on the explanatory factors related to the addition polymerization reaction, and
the explanatory factors include a plurality of feature values obtained by a clustering analysis of time-series data from a plurality of measurement instruments at a temperature rising process and a dropwise addition process, and
the objective factor includes at least either a nonvolatile content (NV) or a solution viscosity.

6. A non-transitory computer-readable recording medium storing instructions performing prediction related to an addition polymerization reaction by an information processing device that comprises a processor, the instructions causing the processor to execute:
training a prediction model based on actual data comprising a plurality of explanatory factors and an objective factor that are related to the addition polymerization reaction; and
predicting, with the prediction model, the objective factor during the addition polymerization reaction based on the explanatory factors related to the addition polymerization reaction,
the explanatory factors include a plurality of feature values obtained by a clustering analysis of time-series data from a plurality of measurement instruments at a temperature rising process and a dropwise addition process, and
the objective factor includes at least either a nonvolatile content (NV) or a solution viscosity.
